(19) United States
(12) Patent Application Publication
(10) Pub. No.: US 2024/0390970 A1
(43) Pub. Date: Nov. 28, 2024

(54) STENT FOR USE IN INTESTINAL TRACT, AND WEAVING METHOD THEREFOR

(71) Applicants: MICROPORT UROCARE (JIAXING) CO., LTD., Jiaxing, Zhejiang (CN); MICROPORT UROCARE (SHANGHAI) CO., LTD., Shanghai (CN)

(72) Inventors: Ran GENG, Shanghai (CN); Jiabo WANG, Shanghai (CN); Xiangyang ZHANG, Shanghai (CN); Zhenzhong FU, Shanghai (CN)

(21) Appl. No.: 18/695,186

(22) PCT Filed: Aug. 23, 2022
(86) PCT No.: PCT/CN2022/114256

§ 371 Date: Mar. 25, 2024

(30) Foreign Application Priority Data
Sep. 24, 2021 (CN) 202111119773.8

(51) Int. Cl. B21F 15/04; A61F 2/90

(52) U.S. Cl. CPC B21F 15/04; A61F 2/90; A61F 2230/0054; A61F 2240/001

(57) ABSTRACT

A stent for use in the intestinal tract and a weaving method therefor. A head section and a tail section of the stent are both weaved by a process comprising: moving a first wire in a first V-shaped bending manner between two adjacent rows of positioning pins until all or half of the positioning pins in a first layer region or in a third layer region are wound, and in the case where half of the positioning pins in the first layer region or in the third layer region are wound, winding all the remaining positioning pins in the first layer region or in the third layer region with a second wire.

### CROSS REFERENCE TO RELATED DISCLOSURE

The present application claims priority to Chinese Patent Disclosure with No. 202111119773.8, entitled “Stent for Use in Intestinal Tract, and Weaving Method Therefor”, and filed on Sep. 24, 2021, the content of which is expressly incorporated herein by reference in its entirety.

### TECHNICAL FIELD

The present disclosure relates to the field of medical devices, and particularly to a stent for use in an intestinal tract, and a weaving method Thereof.

### BACKGROUND

Common weaved stents on the market include cross-type and hook-type woven stents.

The stent of the cross-type structure is formed by spirally winding and weaving a wire in a circumferential direction of a mold. When reaching a top end of the stent, the wire is bent in an opposite direction at a certain angle. In an area between both two ends of the stent, the wire does not bend in the opposite direction. The stent of the cross-type structure has the advantage of good radial support, but has a large shortening rate (i.e., the length of the stent is significantly reduced after the stent is released from the sheath and expanded), and a lower flexibility, (i.e., the greater the external force required to bend the stent, the lower the flexibility of the stent, which is unable to adapt to different curvature of the intestinal tract.

The stent of the hook-type structure is wound around from one end of the stent to the other end in the form of repeated bending, and is connected to each other by a “V”-shaped structure formed by the bending. The stent of the hook-type structure has the disadvantage of lower radial support, but has the advantage of small shortening rate (i.e., the length of the stent is not significantly reduced after the stent is released from the sheath and expanded), and good flexibility (i.e., the smaller the force required to bend the stent, the better the flexibility, which can be adapted to different curvature of the intestinal tract).

As for the performance of the stent, insufficient radial support may affect the support effect of the stent, and the stent cannot provide an effective support force. The larger shortening rate may easily cause the stent to be severely deformed and displaced under the intestinal tract movement, thereby causing functional failure of the stent and leading to a series of complications. The stent with insufficient flexibility cannot be adapted to different curvatures of the intestinal environment and may easily lead to damage to the intestinal wall.

Therefore, the development of a stent with good radial support, a small shortening rate and good flexibility is a current problem to be studied.

### SUMMARY

In view of this, it is necessary to provide a stent for use in an intestinal tract and a weaving method thereof.

A weaving method for a stent used in an intestinal tract, applied to a cylindrical clamp, the cylindrical clamp being provided with a first layer area, a second layer area and a third layer area arranged sequentially in an axial direction, the first layer area and the third layer area each including at least two rows of positioning pins, the second layer area including at least one row of positioning pins, each row of positioning pins in the first layer area, the second layer area, and the third layer area including at least four positioning pins uniformly distributed in a circumferential direction of the cylindrical clamp, the stent for use in the intestinal tract including a head segment, a main body and a tail segment connected in sequence, the weaving method including:
a weaving process of the head segment including: bending and moving a first wire in a first V-shape between two adjacent rows of positioning pins in the first layer area until all the positioning pins in the first layer area are fully wound around or half of the positioning pins in the first layer area are wound around; and allowing a second wire to wind around the remaining positioning pins in the first layer area in a case that half of the positioning pins in the first layer area are wound around by the first wire;
a weaving process of the main body including: bending and moving the first wire in a second V-shape between every two adjacent rows of positioning pins in an area from the last row of positioning pins in the first layer area, the second layer area, to the first row of the positioning pins in the third layer area, until all the positioning pins in the area from the last row of positioning pins in the first layer area, the second layer area, to the first row of the positioning pins in the third layer area are fully wound around; and
a weaving processing of the tail segment including: with the same movement mode as the first wire in the weaving process of the head segment, allowing the first wire to wind around all or half of the positioning pins in the third layer area; and
in the case that half of the positioning pins in the third layer area are wound around by the first wire, allowing the second wire to wind around the remaining positioning pins in the third layer area;
when the first wire or the second wire completes the weaving of adjacent A-th row of positioning pins and B-th row of positioning pins, and the first wire or the second wire returns onto the A-th row of positioning pins, the first wire or the second wire winds around a weaved path between the A-th row of positioning pins and the B-th row of positioning pins, and is moved to the B-th row of positioning pins to form a reinforcing rib, and then starts to move between adjacent B-th row of positioning pins and C-th row of positioning pins; and
when there exists two opposite V-shapes at the same positioning pin, first wires or second wires of the two opposite V-shapes are interlocked with each other.

A stent for use in an intestinal tract, the stent is formed by weaving with the above-mentioned weaving method.

### BRIEF DESCRIPTION OF THE DRAWINGS

The accompanying drawings that form a part of the present disclosure are used for providing further understanding of the present disclosure. The exemplary embodiments of the present disclosure and the description thereof are used for explaining the present disclosure and do not constitute an improper limitation on the present disclosure.

In order to more clearly illustrate the technical solution in the embodiment of the present disclosure, the accompanying drawings required in the description of the embodiments will be briefly introduced below. Obviously, the drawings in the following description are merely some embodiments of the present disclosure. The ordinary skilled in the art can obtain other drawings according to these drawings without any creative efforts.

FIGS. 1 to 13 are flow charts of a weaving method for a stent used in an intestinal tract according to an embodiment I.

FIGS. 14 to 23 are flow charts of a weaving method for a stent used in an intestinal tract according to an embodiment II.

FIG. 24 shows an embodiment in which two opposite V-shaped wires are provided at the same positioning pin, and the two opposite V-shaped first wires or second wires are interlocked with each other.

FIGS. 25 and 26 are enlarged views of a winding mode of a reinforcing rib.

### DETAILED DESCRIPTION

In order to make the above purpose, features and advantages of the present disclosure more obvious and easier to understand, the specific implementation modes of the present disclosure will be described in detail below with reference to the accompanying drawings. In the following description, numerous specific details are set forth in order to provide thorough understanding of the present disclosure. However, the present disclosure may also be implemented in many other modes different from those described here. Those skilled in the art can make similar improvements without departing from the concept of the present disclosure. Therefore, the present disclosure is not limited by the specific embodiments disclosed below.

In an implementation mode, a weaving method for a stent used in an intestinal tract is provided, which uses a cylindrical clamp. The cylindrical clamp is provided with a first layer area, a second layer area and a third layer area arranged sequentially in an axial direction. The first layer area, the second layer area and the third layer area are fixedly or detachably provided with a number of positioning pins.

Specifically, the number of rows of positioning pins in the first layer area is greater than or equal to 2. Each row in the first layer area includes at least 4 positioning pins, and the positioning pins are uniformly distributed in the circumferential direction of the cylindrical clamp. Rows of positioning pins in the first layer area are aligned with each other. The number of rows of positioning pins in the second layer area is greater than or equal to 1, the number of positioning pins in each row in the second layer area is the same as the number of positioning pins in each row in the first layer area, and two adjacent rows of positioning pins are staggered from each other. For example, the number of rows of positioning pins in the second layer area is two, and the two rows of positioning pins are staggered from each other. Alternatively, the number of rows of positioning pins in the second layer area is greater than or equal to 3, two adjacent rows of positioning pins are staggered from each other, and an upper and lower rows of positioning pins adacent to each row of positioning pins are aligned with each other. The number of rows of positioning pins in the third layer area is greater than or equal to 2, the number of positioning pins in each row in the third layer area is the same as the number of positioning pins in each row in the first layer area, and adjacent rows of positioning pins in the third layer area are aligned with each other. Rows of positioning pins in the third layer area and rows of positioning pins in the first layer area are also aligned with each other correspondingly.

Alternatively, the number of rows of positioning pins in the first layer area is greater than or equal to 2. Each row of the first layer area is provided with at least 4 positioning pins, and the positioning pins are uniformly distributed in the circumferential direction of the cylindrical clamp. Two adjacent rows of positioning pins from the rows of positioning pins in the first layer area are staggered from each other, and the upper and lower rows of positioning pins adjacent to each row of positioning pins are aligned with each other. The number of rows of positioning pins in the second layer area is greater than or equal to 1, the number of positioning pins in each row in the second layer area is the same as the number of positioning pins in each row in the first layer area, and two adjacent rows of positioning pins in the second layer area are staggered from each other. For example, the number of rows of positioning pins in the second layer area is two, and the two rows of positioning pins are staggered from each other. Alternatively, the number of rows of positioning pins in the second layer area is greater than or equal to 3, two adjacent rows of positioning pins are staggered from each other, and the upper and lower rows of positioning pins adjacent to each row of positioning pins are aligned with each other. The number of rows of positioning pins in the third layer area is greater than or equal to 2, the number of positioning pins in each row in the third layer area is the same as the number of positioning pins in each row in the first layer area, two adjacent rows of positioning pins in the rows of positioning pins in the third layer area are staggered from each other, and two rows of positioning pins on opposite sides of each row of positioning pins are aligned with each other.

The stent used in the intestinal tract may include a head segment, a main body, and a tail segment that are connected in sequence. The weaving method may include the following steps.

A weaving process of the head segment may include: the first wire is bent and moved in a first V-shape between two adjacent rows of positioning pins in the first layer area until all the positioning pins in each row in the first layer area are fully wound around or half of the positioning pins in the first layer area are wound around. In the case that half of the positioning pins in the first layer area are wound around by the first wire, the second wire is allowed to wind around the remaining positioning pins in the first layer area. When the number of turns of the first wire winding between two adjacent rows of positioning pins is greater than or equal to 3, the first wire, starting from the third turn of weaving, is interweaved up and down around a weaved path in a previous turn or two previous turns. Specifically, a spatial distribution of up and down interweaving needs to follow the principle of uniformity and symmetry. The interweaving can make the weaved wire structure of the stent uniform and tight, and no obvious gaps, bouncing off, deviations, etc., may occur after shaping.

Specifically, the step that in the case that half of the positioning pins in each row in the first layer area are wound around by the first wire, the second wire winds the remaining positioning pins in the first layer area may include the following steps.

The second wire is bent and moved in the first V-shape between two adjacent rows of positioning pins in the first layer area, starting from any positioning pin that the first wire does not pass through, until all the positioning pins in the first layer area are wound around. When the second wire moves between every two adjacent rows of positioning pins, the second wire is interweaved up and down around the weaved path of the first wire. The interweaving can make the weaved wire structure of the stent uniform and tight, and no obvious gaps, bouncing off, deviations, etc., may occur after shaping.

A weaving process of the main body may include: the first wire is bent and moved in a second V-shape between every two adjacent rows of positioning pins in an area from the last row of positioning pins in the first layer area, the second layer area, to the first row of the positioning pins in the third layer area, until all the positioning pins in the area from the last row of positioning pins in the first layer area, the second layer area, to the first row of the positioning pins in the third layer area are fully wound around.

A weaving processing of the tail segment may include: with the same movement mode as the first wire in the weaving process of the head segment, the first wire winds all or half of the positioning pins in each row in the third layer area; in the case that half of the positioning pins in the third layer area are wound around by the first wire, the second wire winds around the remaining positioning pins in the third layer area. When the number of turns of the first wire between every two adjacent rows of positioning pins is greater than or equal to 3, the first wire, starting from the third turn of weaving, is interweaved up and down around the weaved path in a previous turn or two previous turns. Specifically, the spatial distribution of up and down interweaving needs to follow the principle of uniformity and symmetry. The interweaving can make the weaved wire structure of the stent uniform and tight, and no obvious gaps, bouncing off, deviations, etc., may occur after shaping.

The step that in the case that half of the positioning pins in each row in the third layer area are wound around by the first wire, the second wire winds the remaining positioning pins in the third layer area may specifically include:
the second wire is bent and moved in the first V-shape between two adjacent rows of positioning pins in the third layer area with starting from any positioning pin that the first wire does not pass through, until all the positioning pins in the third layer area are wound around. When the second wire is moved between every two adjacent rows of positioning pins, the second wire is interweaved up and down around the weaved path of the first wire. Specifically, the spatial distribution of up and down interweaving needs to follow the principle of uniformity and symmetry. The interweaving can make the weaved wire structure of the stent uniform and tight, and no obvious gaps, bouncing off, deviations, etc., may occur after shaping.

When the first wire or the second wire completes the weaving of adjacent A-th row of positioning pins and B-th row of positioning pins (completion of weaving specifically refers to the that all positioning pins are wound around or half of the positioning pins are wound around as described above), and the first wire or the second wire returns onto the A-th row of positioning pins, the first wire or the second wire winds around the weaved path between the A-th row of positioning pins and the B-th row of positioning pins and is moved to the B-th row of positioning pins to form a reinforcing rib, and then starts to move between the adjacent B-th row of positioning pins and C-th row of positioning pins. A, B, and C refer to any three adjacent rows of positioning pins. If there exists two opposite V-shapes at the same positioning pin, the first wires or second wires with the two opposite V-shapes are interlocked with each other.

For example, in a weaving method, the numbers of rows in the first layer area and the number of rows in the third layer area are both greater than or equal to 2. The numbers of positioning pins in each row in the first and third layer areas are the same, and adjacent rows are aligned with each other. The first V-shape spans (2m+1) positioning pins located in the same row, where m is an integer greater than 1. That is, the first V-shape spans an odd number of positioning pins. The number of positioning pins in each row is greater than 3, and the number of positioning pins in each row is not a multiple of m. The number of positioning pins in the last row in the first layer area, the number of positioning pins in each row in the second layer area, and the number of positioning pins in the first row in the third layer area are the same, and two adjacent rows of positioning pins are staggered from each other. The second V-shape spans 2m′ positioning pins located in the same row, where m′ is an integer greater than 1. In addition, the number of positioning pins in each row is not equal to k(2m−1), where k is a positive integer. For example, m≥m′. More specifically, m and m′ are respectively equal to 2 or 3, which can ensure the performance of the stent used in the intestinal tract and increase the production efficiency. When the number of positioning pins in each row is an odd number, the first V-shape winds around all positioning pins after 2m turns. When the number of positioning pins in each row is an even number, the first V-shape winds around half of the positioning pins after m turns, where m ranges from 2 to 30.

In another weaving method, the number of rows in the first layer area and the number of rows in the third layer area are both greater than or equal to 2. The numbers of positioning pins in each row in the first, second and third layer areas are the same, and adjacent rows are aligned with each other. The first V-shape spans 2n positioning pins located in the same row, where n is an integer greater than 1. That is, the first V-shape can only span an even number of positioning pins. The second V-shape spans 2n′ positioning pins located in the same row, where n′ is an integer greater than 1. For example, n>n′. More specifically, n and n′ are respectively equal to 2 or 3, which can ensure the performance of the stent used in the intestinal tract and increase the production efficiency. The number of positioning pins in each row is greater than 3, and the number of positioning pins in each row is not equal to k(2n−1) and is not equal to k(2n′−1). The first V-shape winds around all positioning pins after (2n−1) turns.

With the above weaving method for the stent, the weaving processes of the head segment and the tail segment both include that the first wire is bent and moved in the first V-shape between two adjacent rows of positioning pins, until all the positioning pins or half of the positioning pins in the first layer area or in the third layer area are wound around. When half of the positioning pins in the first layer area or in the third layer area are wound around, the second wire winds around the first layer area or the third layer area. The weaving process of the main body includes that the first wire is bent and moved in the second V-shape between every two adjacent rows of positioning pins in the area from the last row in the first layer area, the second layer area, to the first row in the third layer area, until all the positioning pins in the area from the last row in the layer area, the second layer area, to the first row in the third layer area are wound around. Accordingly, the wires interweaved between every two rows of positioning pins can provide radial support, and the interlocked structure of the wires on each row of positioning pins can improve the shortening and flexibility of the stent and overall satisfy the required performance of the intestinal stent. In addition, two wires are adopted to weave the stent and control the weaving path, so that the production process of the stent is simpler and more efficient.

The weaving method may further include that the second wire forms another reinforcing rib parallel to the reinforcing rib, specifically including the following steps.

Starting from any positioning pin that the reinforcement rib does not pass through, the second wire winds around the weaved path of the first wire between rows of positioning pins in a direction parallel to the reinforcement rib to form another positioning pin. The reinforcing ribs axially reinforce the stent and reduce the axial shortening degree of the stent. Specifically, the reinforcing rib and the other reinforcing rib are arranged symmetrically with respect to the axis of the stent. Accordingly, the overall structure of the stent is more stable.

The weaving method may further include that at least one third wire forms at least another reinforcing rib parallel to the reinforcing rib, specifically including the following steps.

When all the positioning pins in the first layer area and in the third layer area are wound around, at least one third wire is further provided, and the third wire is weaved using the same method as the second wire to form a reinforcing rib. The plurality of the reinforcing ribs are uniformly distributed in the circumferential direction. For example, the number of the third wires may be one, two, or more. The reinforcing rib and the reinforcing ribs formed by the first wire and the second wire are uniformly distributed in the circumferential direction, to allow for a more stable overall structure of the stent.

The weaving method for the stent used in the intestinal tract in the present disclosure will be illustrated by two specific embodiments as follows.

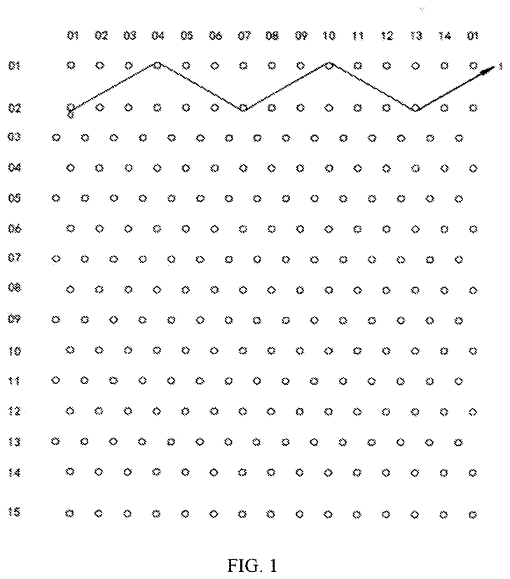

Referring to FIGS. 1 to 13, which are flow charts showing a weaving method for a stent used in an intestinal tract according to the embodiment I. FIGS. 1 to 13 show schematic diagrams of a cylindrical clamp in an expanded state. A number of dots in the figures represent positioning pins. In FIGS. 1 to 13, the number of rows of positioning pins in the first layer area is 2, i.e., rows 01 and 02 arranged longitudinally in FIGS. 1 to 13. Each row is provided with 14 positioning pins, respectively indicated by 01 to 14 arranged transversely in FIGS. 1 to 13. The 14 positioning pins are uniformly distributed in the circumferential direction of the cylindrical clamp, and the two rows of positioning pins in the first layer area are aligned with each other.

The number of rows of positioning pins in the second layer area is 11, respectively shown as rows 03 to 13 arranged longitudinally in FIGS. 1 to 13. Each row is provided with 14 positioning pins, and the 14 positioning pins are uniformly distributed in the circumferential direction of the cylindrical clamp. Among the 11 rows of positioning pins in the second layer area, every two adjacent rows of positioning pins are staggered from each other, and the upper and lower rows of positioning pins adjacent to each row of positioning pins are aligned with each other. That is, the odd-numbered rows of positioning pins in the second layer area are aligned with each other, the even-numbered rows of positioning pins in the second layer area are aligned with each other, but the odd-numbered row of positioning pins and the even-numbered row of positioning pins are staggered from each other.

The number of rows of positioning pins in the third layer area is 2, i.e., respectively shown as rows 14 and 15 arranged longitudinally in FIGS. 1 to 13. Each row is provided with 14 positioning pins, and the 14 positioning pins are uniformly distributed in the circumferential direction of the cylindrical clamp. The two rows of positioning pins in the third layer area are aligned with each other. The two rows of positioning pins in the third layer area are also aligned with the two rows of positioning pins in the first layer area.

The rows of positioning pins in the first layer area are equally spaced. Similarly, the rows of positioning pins in the third layer area are equally spaced. The rows of positioning pins in the second layer area are also equally spaced. However, the spacing between every two rows of positioning pins in the second layer area may be unequal to the spacing between every two rows of positioning pins in the first layer area and in the third layer area. Alternatively, the spacing between every two rows of positioning pins in the second layer area is equal to the spacing between every two rows of positioning pins in the first layer area and in the third layer area.

### Embodiment I

### The Weaving of the First Wire

Referring to FIG. 1, the first wire is weaved from a first initial point (i.e., a position corresponding to the number 0 of the weaved path with an arrow in FIG. 1) in a direction away from the second layer area, and is bent and moved in a large V-shape (i.e., the first V-shape mentioned above) between two adjacent rows of positioning pins in the first layer area, and moved to a first position in FIG. 1 (i.e., a position corresponding to the number 1 of the weaved path with an arrow in FIG. 1) to complete the first turn of weaving in the first layer area. The first initial point is any one of the positioning pins in the second row in the first layer area. In the first implementation mode, the large V-shape spans 7 positioning pins located in the same row.

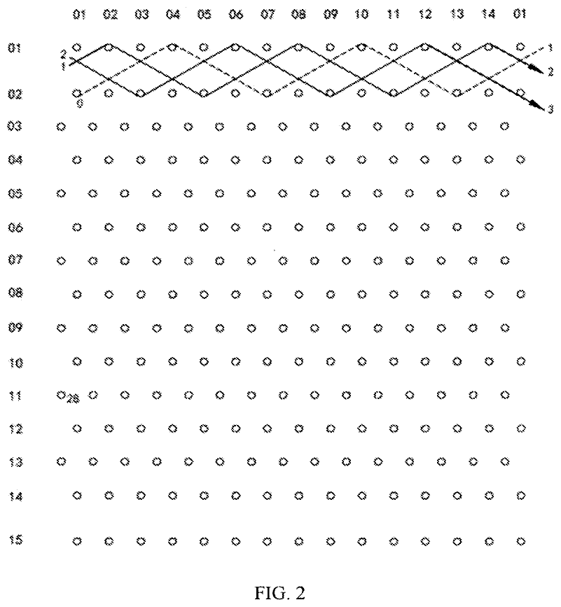

Referring to FIG. 2, the first wire is continuously bent and moved in the large V-shape from the first position to the second position in FIG. 2 (i.e., a position corresponding to the number 2 of the weaved path with an arrow in FIG. 2), to complete the second turn of weaving in the first layer area. The first wire is continuously bent and moved in the large V-shape from the second position to the third position in FIG. 2 (i.e., a position corresponding to the number 3 of the weaved path with an arrow in FIG. 2. At this point, the first wire returns to the first initial point), and accordingly the third turn of weaving in the first layer area is completed, so that half of the positioning pins in the first layer area are wound around. During the third turn of weaving, the first wire is interweaved up and down between the first turn of wire and the second turn of wire. The interweaving can make the weaved wire structure of the stent uniform and tight, and no obvious gaps, bouncing off, deviations, etc., may occur after shaping.

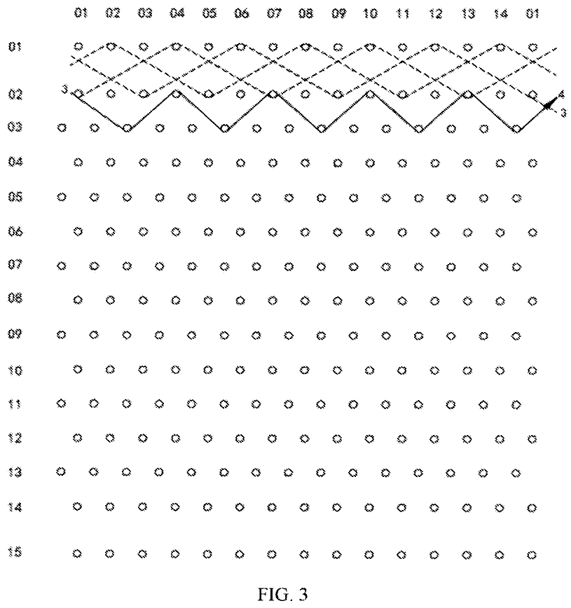

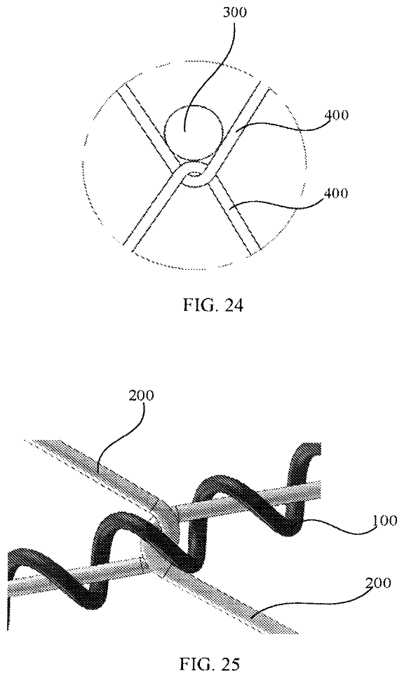

Referring to FIG. 3, the first wire is weaved from the first initial point in a direction towards the third layer area, and is bent and moved in a small V-shape, i.e., the second V-shape mentioned above, between the last row of positioning pins in the first layer area and the first row of positioning pins in the second layer area, and is moved to the fourth position in FIG. 3, i.e., a position corresponding to the number 4 of the weaved path with an arrow in FIG. 3, to complete the first turn of weaving between the last row of positioning pins in the first layer area and the first row of positioning pins in the second layer area. The small V-shape spans 4 positioning pins located in the same row. After the first turn of weaving is completed, two opposite V-shapes 400 are formed for some positioning pins 300, and the first wires of the two opposite V-shapes 400 are interlocked with each other, as shown in FIG. 24.

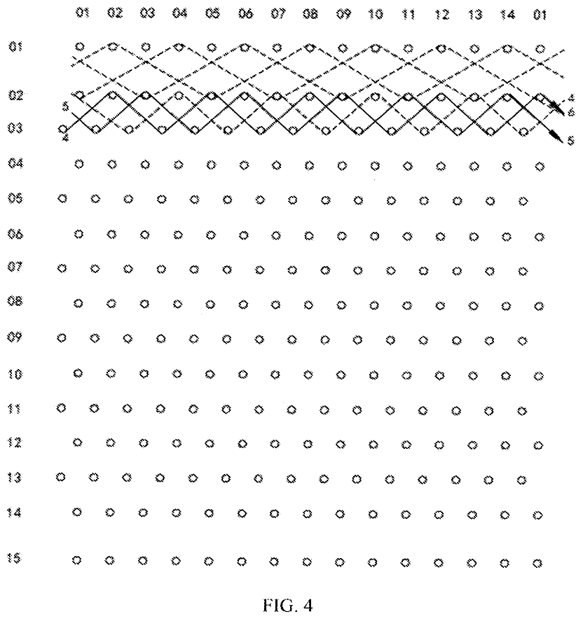

Referring to FIG. 4, the first wire is repeatedly bent and moved in the small V-shape from the fourth position to complete the second turn of weaving between the last row of positioning pins in the first layer area and the first row of positioning pins in the second layer area, and reaches the fifth position in FIG. 3, i.e., a position corresponding to the number 5 of the weaved path with an arrow in FIG. 4, and is continuously moved to complete the third turn of weaving between the last row of positioning pins in the first layer area and the first row of positioning pins in the second layer area, and then reaches the sixth position in FIG. 3, i.e., a position corresponding to the number 6 of the weaved path with an arrow in FIG. 4, at this moment the first wire returns to the first initial point, so that the first wire is fully wound around the last row of positioning pins in the first layer area and the first row of positioning pins in the second layer area. After the second and third turns of weaving are completed, two opposite V-shapes 400 are formed for more positioning pins 300 in the last row in the first layer area, and the first wires of the two opposite V-shapes 400 are interlocked, as shown in FIG. 24.

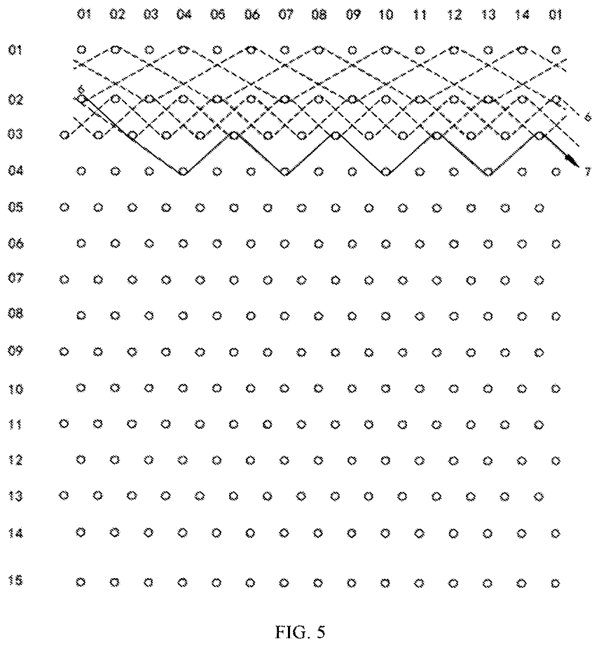

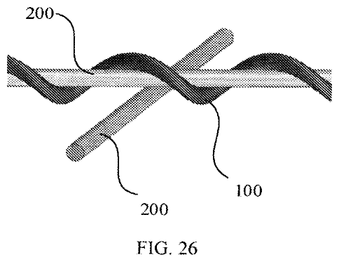

Referring to FIG. 5, starting from the first initial point in the direction towards the third layer area, the first wire winds around the weaved first wire between the last row of positioning pins in the first layer area and the first row of positioning pins in the second layer area mentioned in the previous step, to form a reinforcing rib. That is, the first wire winds around the first weaved wire mentioned in the previous step from the sixth position along the second row of positioning pins in the first layer area, to form the reinforcing rib. For the reinforcing rib 100, exemplary reference can be made to FIGS. 25 and 26, but is not limited to the mode shown in the figures. The reinforcing rib 100 spirally winds around the first weaved wire 200 mentioned in the previous step to form the reinforcing rib 100. As shown in FIG. 25, the reinforcing rib 100 is wound around at two opposite V-shapes of the same positioning pin. As shown in FIG. 26, the reinforcing rib winds around the intersection of the first wires 200, such as the intersection at the small V-shape between the last row of positioning pins in the first layer area and the first row of positioning pins in the second layer area shown in FIG. 5. The reinforcing rib can axially reinforce the stent, thereby reducing the axial shortening degree of the stent.

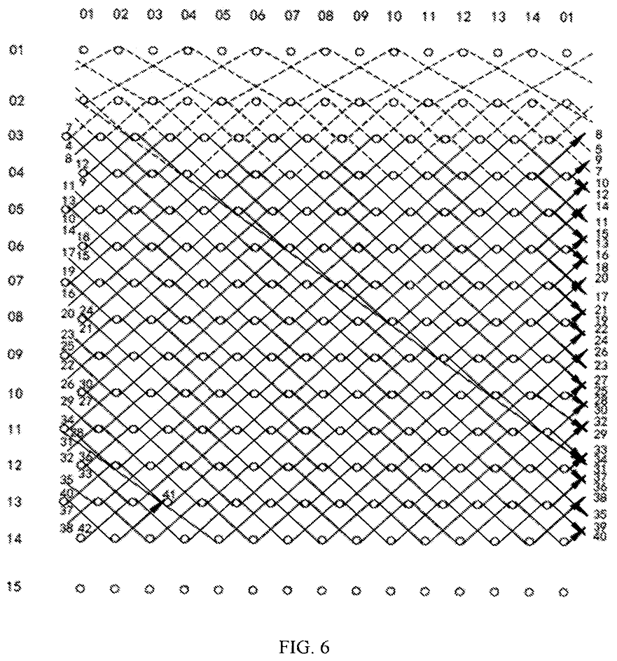

Referring to FIGS. 5 and 6, the steps in FIGS. 4 and 5 are repeated for each row of positioning pins in the second layer area until the last row of positioning pins in the second layer area and the first row of positioning pins in the third layer area are all wound around. That is, the wire is bent and moved in a small V-shape from the initial point in the second layer area to the seventh position in FIG. 5, until the first wire is moved to the 41-st position. At the moment, the positioning pins in the last row in the second layer area and the first row of positioning pins in the third layer area are all fully wound around.

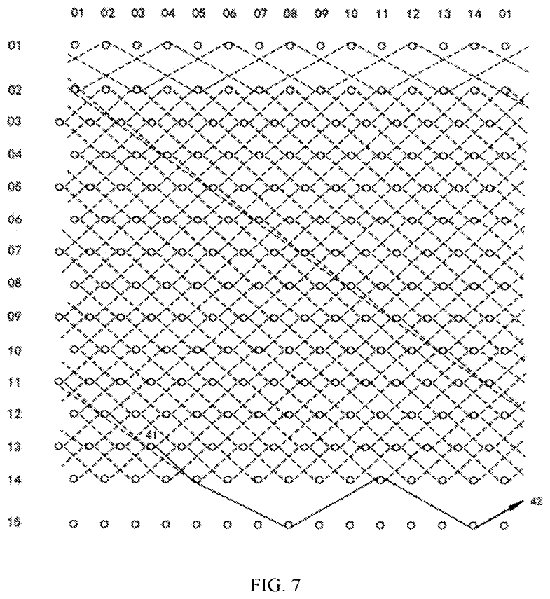

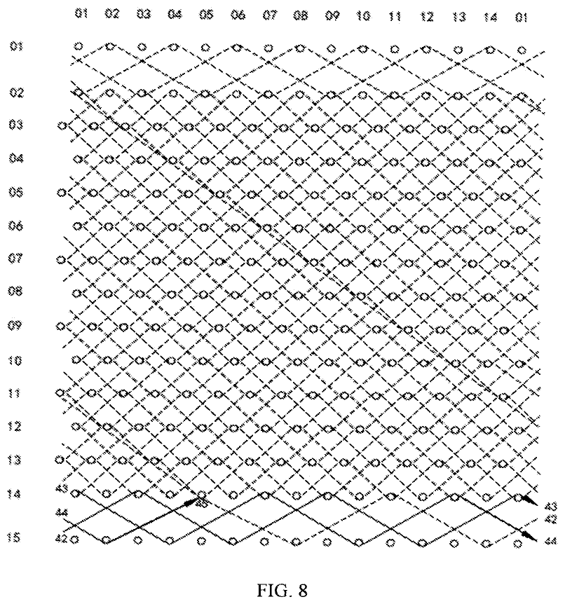

Referring to FIGS. 7 and 8, the first wire winds around the weaved path between the last row of positioning pins in the second layer area and the first row of positioning pins in the third layer area, and is moved to the second initial point of the first row of positioning pins in the third layer area to form the reinforcing rib 100 (the reference can be made to FIGS. 25 and 26 for the structure of the reinforcing rib 100), and then the first wire is weaved from the second initial point of the first row in the third layer area in a direction away from the second layer area. Similar to the steps of weaving in the first layer area, the first wire is bent and moved in the large V-shape between two adjacent rows of positioning pins in the third layer area, until reaching the 45th position, i.e., the second initial point of the first row in the third layer area, so that half of the positioning pins in the third layer area are wound around.

### Weaving of the Second Wire

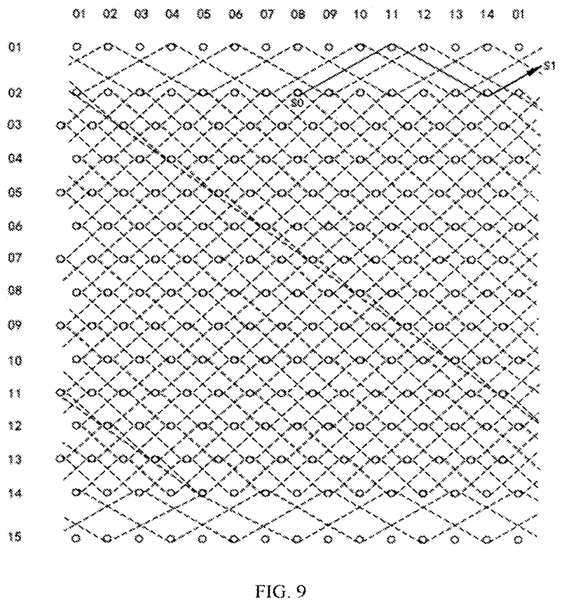

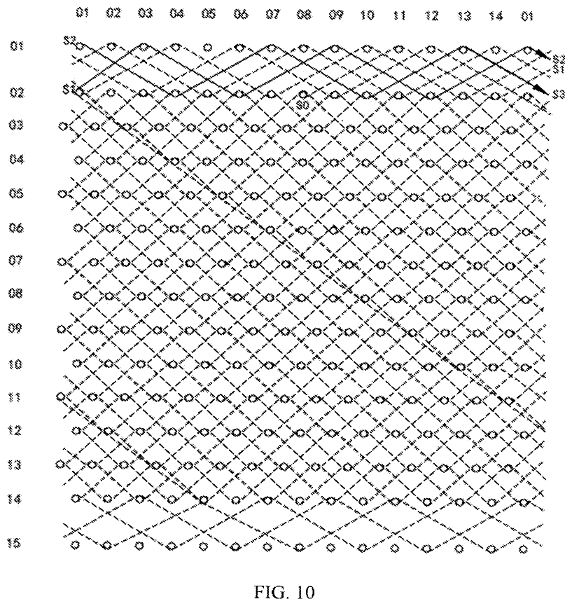

Referring to FIGS. 9 and 10, the second wire is weaved from a relative position of the first initial point of the first wire, i.e., a position corresponding to S0 in FIG. 9, where the relative position of the first initial point refers to a position which is symmetric with the first initial point with respect to the axis and is located in the same row with the first initial point. In the embodiment, the weaving path of the second wire involves S0, S1, . . . , S8, and has the same steps of weaving of the first wire in the first layer area. The second wire is bent and moved in the large V-shape between the two adjacent rows of positioning pins in the first layer area, completes the weaving of the first turn and reaches the position S1, completes the weaving of the second turn and reaches the position S2, and completes the weaving of the third turn and reaches the position S3, until the second wire returns to the relative position (S0) of the first initial point of the first wire, so that all the positioning pins in the first layer area are fully wound around.

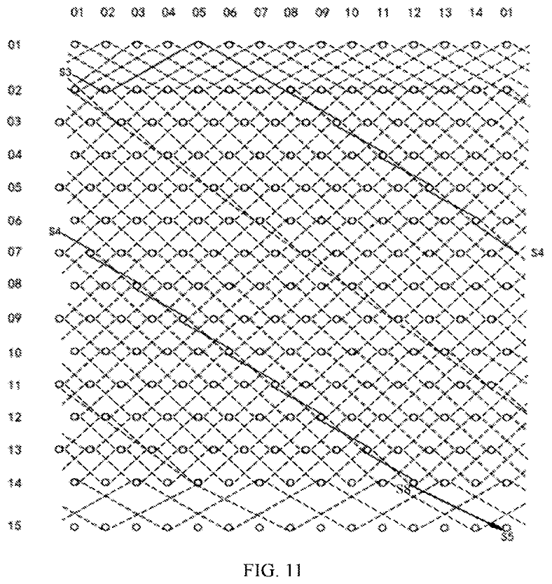

Referring to FIG. 11, starting from the relative position (position S0) corresponding to the first initial point of the first wire, the second wire winds around the weaved paths between the rows of positioning pins in the second layer area along a path parallel to the reinforcing rib in the direction towards the third layer area, until the second wire winds the third initial point (position S8) of the first row in the third layer area. Reference can be made to FIGS. 25 and 26 for the winding method of the reinforcing rib.

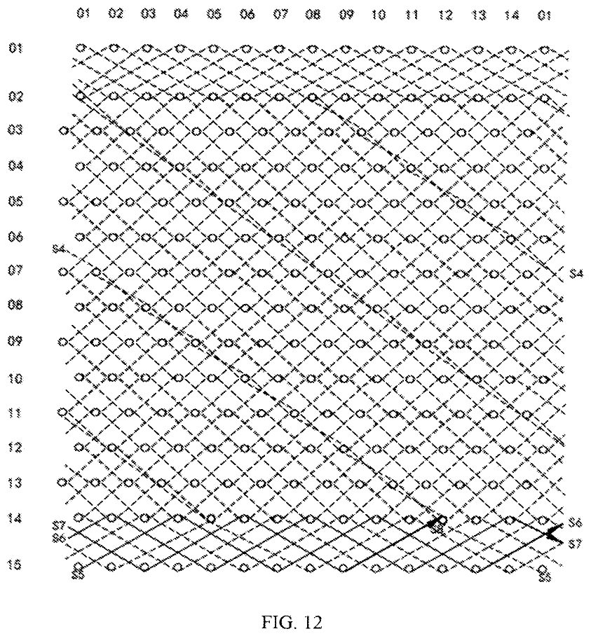

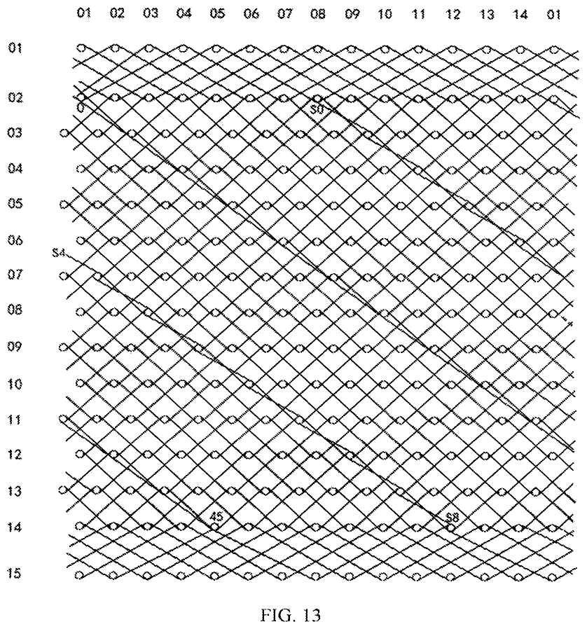

Referring to FIGS. 12 and 13, the second wire is weaved from the third initial point (position S8) of the first row in the third layer area in a direction away from the second layer area. Similar to the weaving steps of the first wire in the first layer area, the second wire is bent and moved in the large V-shape between two adjacent rows of positioning pins in the third layer area, completes the weaving of the first turn and reaches the position S5, completes the weaving of the second turn and reaches the position S6, and completes the weaving of the third turn and reaches the position S7, until the second wire returns to the third initial point position S8 of the first row in the third layer area, so that all the positioning pins in the third layer area are fully wound around. During the weaving process of the stent, if there are two opposite V-shapes 400 at the same positioning pin, the first or second wires of the two opposite V-shapes 400 are interlocked, referring to FIG. 24.

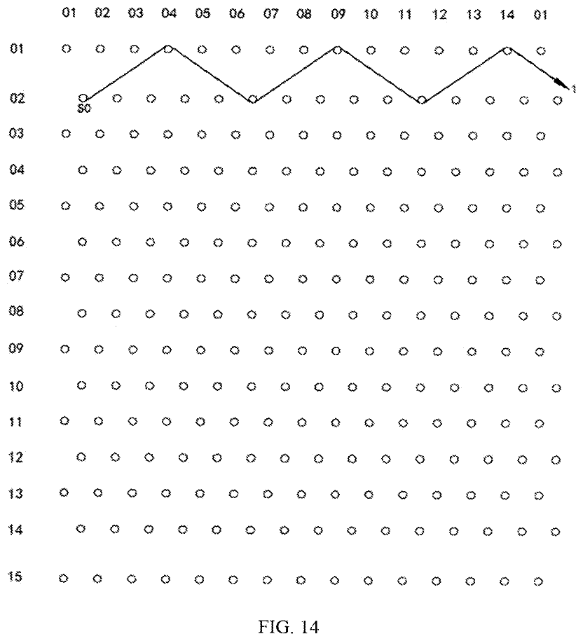

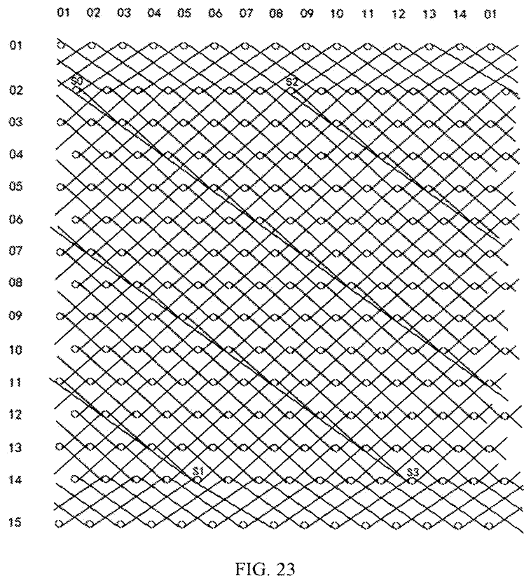

Referring to FIGS. 14 to 23, which are flow charts showing a weaving method for a stent used in an intestinal tract according to the embodiment II. FIGS. 14 to 23 show schematic diagrams of a cylindrical clamp in an expanded state. A number of dots in the figures represent positioning pins. In FIGS. 14 to 23, the number of rows of positioning pins in the first layer area is 2, i.e., the rows 01 and 02 arranged longitudinally in FIGS. 14 to 23. Each row is provided with 14 positioning pins, respectively indicated by 01 to 14 arranged transversely in FIGS. 14 to 23. The 14 positioning pins are uniformly distributed in the circumferential direction of the cylindrical clamp, and two rows of positioning pins in the first layer area are staggered from each other.

The number of the rows of positioning pins in the second layer area is 11, respectively indicated by rows 03 to 13 arranged longitudinally in FIFS. 14 to 23. Each row is provided with 14 positioning pins, and the 14 positioning pins are uniformly distributed in the circumferential direction of the cylindrical clamp. Among the 11 rows of positioning pins in the second layer area, every two adjacent rows of positioning pins are staggered from each other, and the upper and lower rows of positioning pins adjacent to each row of positioning pins are aligned with each other. That is, the odd-numbered rows of positioning pins in the second layer area are aligned with each other, the even-numbered rows of positioning pins in the second layer area are aligned with each other, but the odd-numbered row of positioning pins and the even-numbered row of positioning pins are staggered from each other.

The number of rows of positioning pins in the third layer area is 2, i.e., the rows 14 and 15 arranged longitudinally in FIGS. 14 to 23. Each row is provided with 14 positioning pins, and the 14 positioning pins are uniformly distributed in the circumferential direction of the cylindrical clamp. The two rows of positioning pins in the third layer area are staggered from each other. The first row of positioning pins in the first layer area are aligned with the odd-numbered rows of positioning pins in the second layer area and the second row of positioning pins in the third layer area. The second row of positioning pins in the first layer area are aligned with the even-numbered rows of positioning pins in the second layer area and the first row of positioning pins in the third layer area.

The rows of positioning pins in the first layer area are equally spaced. Similarly, the rows of positioning pins in the third layer area are equally spaced, and the rows of positioning pins in the second layer area are also equally spaced. However, the spacing between rows of positioning pins in the second layer area may be unequal to the spacing between rows of positioning pins in the first layer area and in the third layer area. Alternatively, the spacing between rows of positioning pins in the second layer area is equal to the spacing between rows of positioning pins in the first layer area and in the third layer area.

### Embodiment II

### The Weaving of the First Wire

Referring to FIG. 14, the first wire is weaved from the first initial point, i.e., the position corresponding to S0 of the weaved path with an arrow in FIG. 14, in the direction away from the second layer area, and is bent and moved in a large V-shape, i.e., the first V-shape mentioned above, between two adjacent rows of positioning pins in the first layer area, and is moved to the first position in FIG. 14, i.e., the position corresponding to the number 1 of the weaved path with an arrow in FIG. 14, and completes the weaving of the first turn in the first layer area. The first initial point is any one of the positioning pins in the second row in the first layer area. In the first implementation mode, the large V-shape spans six positioning pins located in the same row.

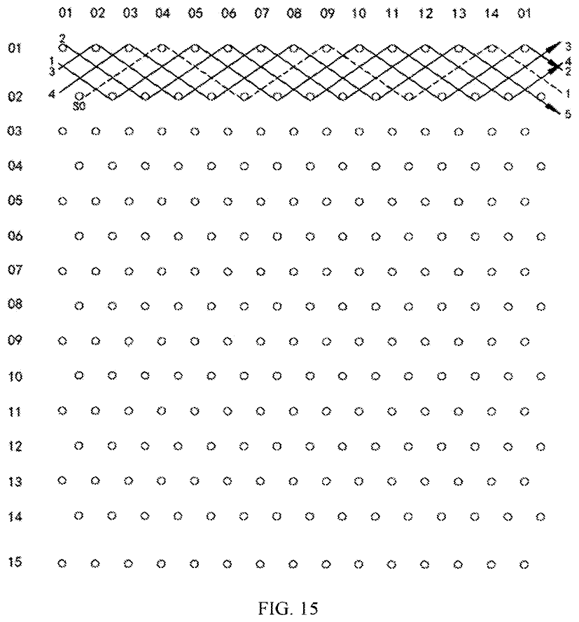

Referring to FIG. 15, the first wire is continuously bent and moved in the large V-shape, i.e., the above-mentioned first V-shape, from the first position and is moved to the second position in FIG. 15 to complete the second turn of weaving in the first layer area. The first wire is continuously bent and moved in the large V-shape from the second position and moved to the third position in FIG. 15, to complete the third turn of weaving in the first layer area. The first wire is continuously bent and moved in the large V-shape from the third position and moved to the fourth position in FIG. 15, to complete the fourth turn of weaving in the first layer area. The first wire is continuously bent and moved in the large V-shape from the fourth position and moved to the fifth position in FIG. 15, and at the moment the first wire returns to the first initial point, to complete the fifth turn of weaving in the first layer area, so that all the positioning pins in the first layer area are fully wound around. During the weaving process starting from the third turn, the first wire is interweaved up and down around a weaved path in a previous turn or two previous turns. The interweaving can make the weaved wire structure of the stent uniform and tight, and no obvious gaps, bouncing off, deviations, etc., may occur after shaping.

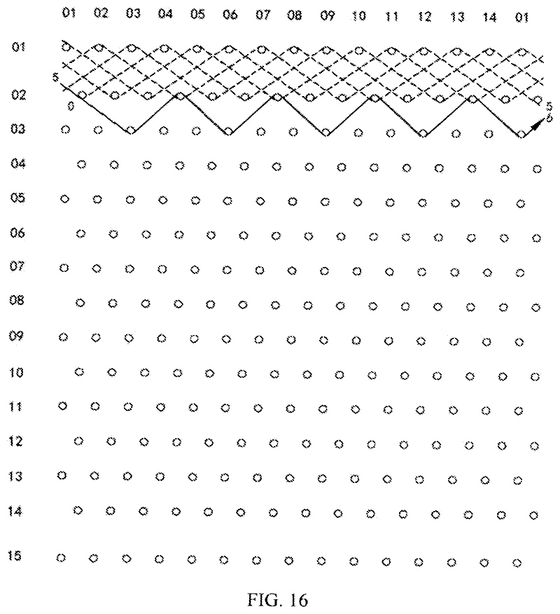

Referring to FIG. 16, the first wire is weaved from the first initial point in the direction towards the third layer area, and is bent and moved in a small V-shape between the last row of positioning pins in the first layer area and the first row of positioning pins in the second layer area, and is moved to the sixth position in FIG. 16. The small V-shape spans 4 positioning pins located in the same row. When the first wire is moved to the sixth position in FIG. 16, there are two opposite V-shapes 400 formed for some of the positioning pins in the last row in the first layer area. The first wires of the two opposite V-shapes 400 are interlocked with each other, as shown in FIG. 24.

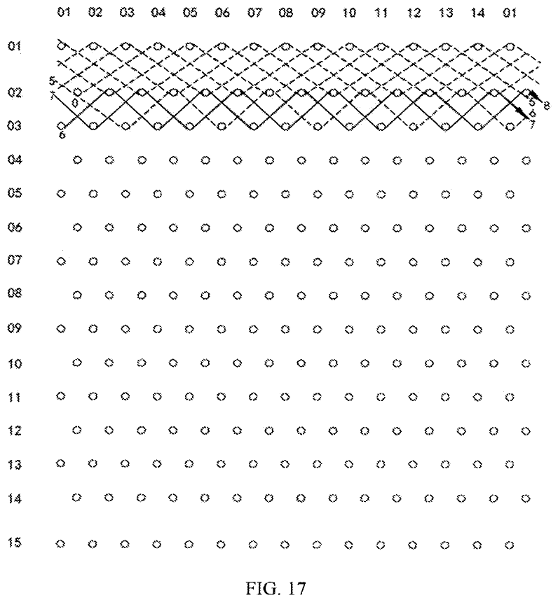

Referring to FIG. 17, the first wire is repeatedly bent and moved in the small V-shape, i.e., the above-mentioned second V-shape, from the sixth position until the first wire is moved to the eighth position, i.e., the first initial point, so that the first wire winds around the last row of positioning pins in the first layer area and the first row of positioning pins in the second level area. There are two opposite V-shapes at more positioning pins in the last row in the first layer area, and the first wires of the two opposite V-shapes are interlocked with each other.

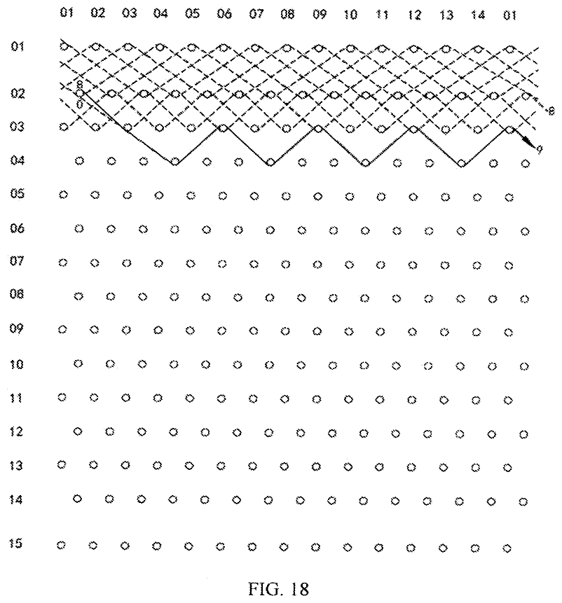

Referring to FIG. 18, starting from the eighth position, i.e., the first initial point, in the direction towards the third layer area, the first wire winds around the first weaved wire between the last row of positioning pins in the first layer area and the first row of positioning pins in the second layer area mentioned in the previous step to form a reinforcing rib. That is, the first wire winds around the first weaved wire mentioned in the previous step from the eighth position along the second row of positioning pins in the first layer area to form the reinforcing rib. The reinforcing rib can axially reinforce the stent, thereby reducing the axial shortening degree of the stent. Reference can be made to FIGS. 25 and 26 for the reinforcing rib 100. The reinforcing rib 100 spirally winds around the first weaved wire 200 mentioned in the previous step to form the reinforcing rib. As shown in FIG. 25, the reinforcing rib winds around at two opposite V-shapes at the same positioning pin. As shown in FIG. 26, the reinforcing rib winds around at the intersection of the first wires, such as at the intersection of the small V-shape between the last row of positioning pins in the first layer area and the first row of positioning pins in the second layer area shown in FIG. 5.

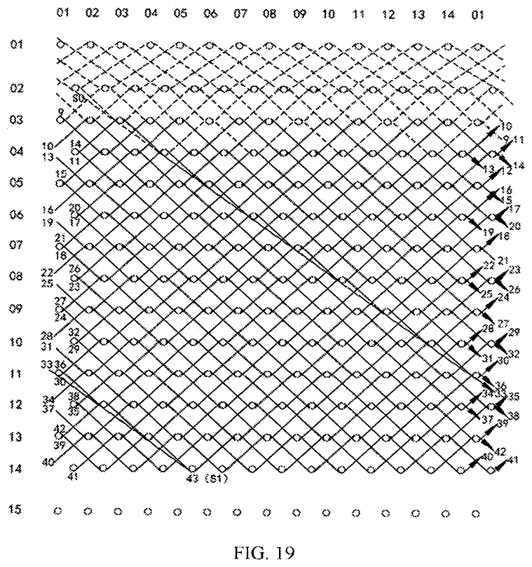

Referring to FIGS. 18 and 19, the steps shown in FIGS. 17 and 18 are repeated for each row of positioning pins in the second layer area until the last row of positioning pins in the second layer area and the first row of positioning pins in the third layer area are fully wound around. That is, the first wire is bent and moved in the small V-shape from the initial point in the second layer area to the ninth position in FIG. 18 until the first wire is moved to the 43-rd position (S1), and at this moment, the last row of positioning pins in the second layer area and the first row of positioning pins in the third layer area are fully wound around.

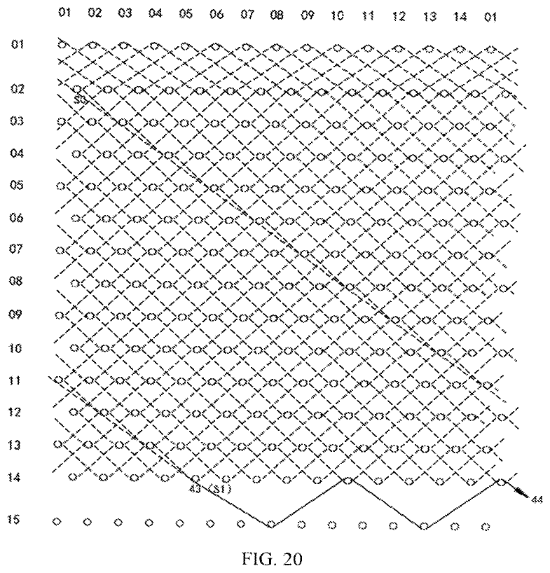

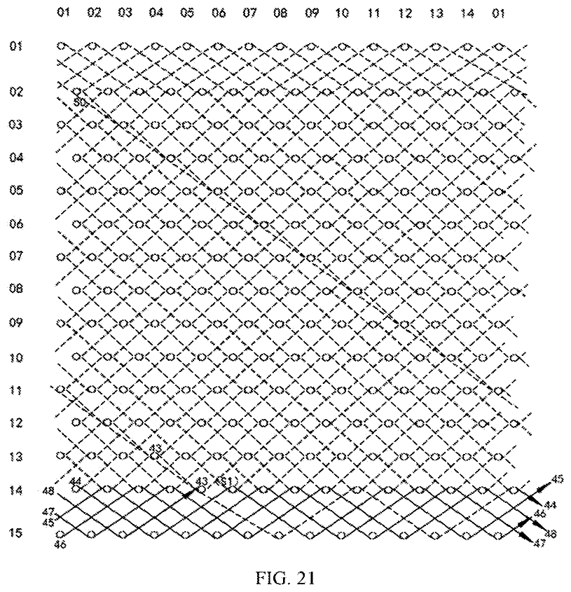

Referring to FIGS. 20 and 21, the first wire winds around the weaved path between the last row of positioning pins in the second layer area and the first row of positioning pins in the third layer area, and is moved to the second initial point of the first row of positioning pins in the third layer area to form a reinforcing rib, and then the first wire is weaved from the second initial point of the first row of positioning pins in the third layer area in a direction away from the second layer area, similar to the steps of weaving in the first layer area, the first wire is bent and moved in the large V-shape between two adjacent rows of positioning pins in the third layer area, until the first wire reached the 44th position, and the first wire is bent and moved in the large V-shape between two adjacent rows of positioning pins in the third layer area until the 45th to 48th positions, and finally returns to the 43rd position, i.e., the second initial point of the first row in the third layer area, so that all the positioning pins in the third layer area are fully wound around.

### Weaving of the Second Wire

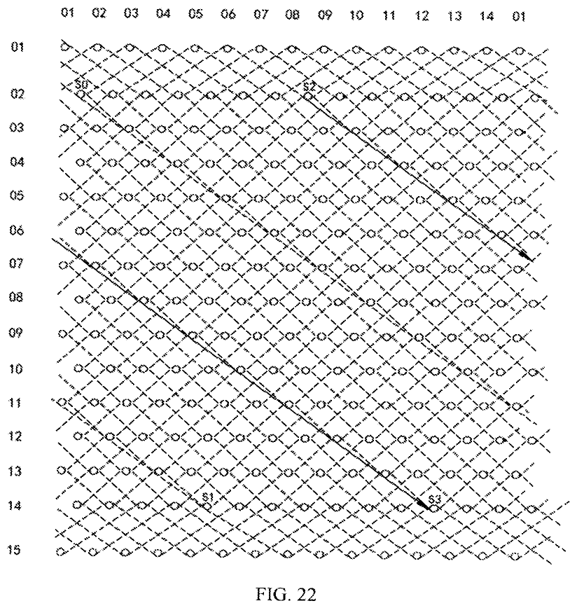

Referring to FIG. 22, the second wire is weaved from a relative position of the first initial point of the first wire, i.e., the position corresponding to S2 of the weaved path with an arrow in FIG. 22, and the relative position of the first initial point refers to a position which is symmetric with the first initial point with respect to the axis and is located in the same row as the first initial point. The second wire winds around the weaved path of the first wire between rows of positioning pins in a direction parallel to the reinforcing rib to form another reinforcing rib mentioned above, and finally is fixed in the last row in the second layer area, i.e., position S3. Reference can be made to FIGS. 25 and 26 for the winding method of the reinforcing rib. During the weaving process of the stent, if there are two opposite V-shapes 400 at the same positioning pin 300, the first wires or the second wires of the two opposite V-shapes 400 are interlocked with each other, as shown in FIG. 24.

The present disclosure further provides a stent for use in an intestinal tract, which is formed by weaving with the weaving method described in the above-mentioned embodiment I or II.

The technical limitations in the above-described embodiments can be combined in any way. In order to simplify the description, all possible combinations of the technical limitations in the above-described embodiments are not described. However, as long as there is no contradiction in the combination of these technical limitations, all should be considered as falling within the scope of the present disclosure.

The above-described embodiments only express several implementation modes of the present disclosure, and the description is relatively specific and detailed, but should not be construed as limiting the scope of the patent disclosure. It should be noted that, those of ordinary skill in the art can make several modifications and improvements without departing from the concept of the present disclosure, and these all fall within the protection scope of the present disclosure. Therefore, the protection scope of the present disclosure should be subject to the appended claims.

1. A weaving method for a stent used in an intestinal tract, applied to a cylindrical clamp, the cylindrical clamp being provided with a first layer area, a second layer area and a third layer area arranged sequentially in an axial direction, the first layer area and the third layer area each comprising at least two rows of positioning pins, the second layer area comprising at least one row of positioning pins, each row of positioning pins in the first layer area, the second layer area, and the third layer area comprising at least four positioning pins uniformly distributed in a circumferential direction of the cylindrical clamp, the stent for use in the intestinal tract comprising a head segment, a main body and a tail segment connected in sequence, the weaving method comprising:
a weaving process of the head segment comprising: bending and moving a first wire in a first V-shape between two adjacent rows of positioning pins in the first layer area until all the positioning pins in the first layer area are fully wound around or half of the positioning pins in the first layer area are wound around; and allowing a second wire to wind around the remaining positioning pins in the first layer area in a case that half of the positioning pins in the first layer area are wound around by the first wire;
a weaving process of the main body comprising: bending and moving the first wire in a second V-shape between every two adjacent rows of positioning pins in an area from the last row of positioning pins in the first layer area, the second layer area, to the first row of the positioning pins in the third layer area, until all the positioning pins in the area from the last row of positioning pins in the first layer area, the second layer area, to the first row of the positioning pins in the third layer area are fully wound around; and
a weaving processing of the tail segment comprising: with the same movement mode as the first wire in the weaving process of the head segment, allowing the first wire to wind around all or half of the positioning pins in the third layer area; and in the case that half of the positioning pins in the third layer area are wound around by the first wire, allowing the second wire to wind around the remaining positioning pins in the third layer area;
wherein, when the first wire or the second wire completes the weaving of adjacent A-th row of positioning pins and B-th row of positioning pins, and the first wire or the second wire returns onto the A-th row of positioning pins, the first wire or the second wire winds around a weaved path between the A-th row of positioning pins and the B-th row of positioning pins, and is moved to the B-th row of positioning pins to form a reinforcing rib, and then starts to move between adjacent B-th row of positioning pins and C-th row of positioning pins; and
wherein when there exists two opposite V-shapes at the same positioning pin, first wires or second wires of the two opposite V-shapes are interlocked with each other.

2. The weaving method for the stent used in the intestinal tract according to claim 1, wherein allowing the second wire to wind around the remaining positioning pins in the first layer area and in the third layer area when at least half of the positioning pins in the first layer area and in the third layer area are wound around comprises:
bending and moving the second wire in the first V-shape between two adjacent rows of positioning pins in the first layer area with starting from a positioning pin that the first wire does not pass through, until all the positioning pins in the first layer area are wound around; and
bending and moving the second wire in the first V-shape between two adjacent rows of positioning pins in the third layer area with starting from a positioning pin that the first wire does not pass through, until all the positioning pins in the third layer area are wound around.

3. The weaving method for the stent used in the intestinal tract according to claim 2, wherein when the second wire is moved between every two adjacent rows of positioning pins, the second wire is interweaved up and down around the weaved path of the first wire.

4. The weaving method for the stent used in the intestinal tract according to claim 1, wherein when the number of turns of the first wire winding between every two adjacent rows of positioning pins is greater than or equal to 3, the first wire, starting from the third turn of weaving, is interweaved up and down around a weaved path in a previous turn or two previous turns.

5. The weaving method for the stent used in the intestinal tract according to claim 1, further comprising: allowing the second wire to form another reinforcing rib parallel to the reinforcing rib, which comprises:
allowing the second wire to, with starting from a positioning pin that the reinforcement rib does not pass through, wind around a weaved path of the first wire between rows of positioning pins in a direction parallel to the reinforcement rib to form another reinforcing rib.

6. The weaving method for the stent used in the intestinal tract according to claim 5, wherein the reinforcing rib and the another reinforcing rib are arranged symmetrically with respect to an axis of the stent.

7. The weaving method for the stent used in the intestinal tract according to claim 6, wherein when all the positioning pins in the first layer area and the third layer area are fully wound around, at least one third wire is further provided, the third wire is weaved to form a reinforcing rib using the same weaving method as the second wire, and a plurality of the reinforcing ribs are uniformly distributed in the circumferential direction.

8. The weaving method for the stent used in the intestinal tract according to claim 1, wherein the number of positioning pins in each row in the first layer area is the same as the number of positioning pins in each row in the third layer area, and adjacent rows of positioning pins are aligned with each other;
the first V-shape spans (2m+1) positioning pins located in the same row, where m is an integer greater than 1;
the number of positioning pins in the last row in the first layer area, the number of positioning pins in each row in the second layer area, and the number of positioning pins in the first row in the third layer area are the same, and two adjacent rows of positioning pins are staggered from each other; and
the second V-shape spans 2m′ positioning pins located in the same row, where m′ is an integer greater than 1.

9. The weaving method for the stent used in the intestinal tract according to claim 8, wherein m≥m′.

10. The weaving method for the stent used in the intestinal tract according to claim 8, wherein m and m′ are respectively equal to 2 or 3.

11. The weaving method for the stent used in the intestinal tract according to claim 1, wherein the number of positioning pins in each row in the first layer area, the number of positioning pins in each row in the second layer area, and the number of positioning pins in each row in the third layer area are the same, and two adjacent rows of positioning pins are staggered from each other;
the first V-shape spans 2n positioning pins located in the same row, where n is an integer greater than 1; and
the second V-shape spans 2n′ positioning pins located in the same row, where n′ is an integer greater than 1.

12. The weaving method for the stent used in the intestinal tract according to claim 11, wherein n>n′.

13. The weaving method for the stent used in the intestinal tract according to claim 11, wherein n and n′ are respectively equal to 2 or 3.

14. A stent for use in an intestinal tract, wherein the stent is formed by weaving with the weaving method of claim 1.
